(19) United States
(12) Patent Application Publication
(10) Pub. No.: US 2014/0341996 A1
(43) Pub. Date: Nov. 20, 2014

(54) Nanoengineering of Functionalized Polymers and Its Manufacturing and Formulation Methods for Personalized Cancer Therapies

(71) Applicant: Kumarpal A. Shah, Searingtown, NY (US)

(72) Inventor: Kumarpal A. Shah, Searingtown, NY (US)

(21) Appl. No.: 14/276,130

(22) Filed: May 13, 2014

Related U.S. Application Data
(60) Provisional application No. 61/824,812, filed on May 17, 2013.

(51) Int. Cl. A61K 31/795; A61K 9/14

(52) U.S. Cl. CPC A61K 31/795; A61K 9/14
USPC 424/489; 424/78.17; 424/493

(57) ABSTRACT

Nanoengineering of inert polymers to develop functionalized sulfonated polymers to harness the power of Alternate complement system to stimulate and amplify cytotoxic potentials of classical and lectin based complement system Manufacturing functionalized sulfonated polymer to better penetrate tumor microenvironment, actively target various cancer antigens in conjunction with monoclonal antibodies in a safe way to inhibit host inflammatory reactions while maximizing cytotoxic potentials. The methods provide nanopolymers to safely maximize the cytotoxic potential of existing and evolving cancer therapies. Combining nanopolymers with existing and evolving cancer drugs to provide personalized cancer therapies.

### CROSS-REFERENCE TO RELATED APPLICATIONS

The invention claims priority from U.S. Provisional Application Ser. No. 61/824,812 filed on May 17, 2013, entitled MANUFACTURING AND FORMULATION METHODS OF SULFONIC POLYMERS FOR TARGETED CANCER THERAPY, the entire contents of which is incorporated herein by reference thereto.

### BACKGROUND OF THE INVENTION

1. Field of the Invention

The invention relates to nanoengineering of functionalized polymers and its manufacturing and formulation methods for personalized cancer therapies.

2. the Prior Art

The Fundamentals of immunology have contributed greatly to the development personalized cancer therapeutics. Three dimensionally, the concept of fundamentals of immunology utilizes the power of host classical complement system to engage tumor antigens with monoclonal antibodies. Antigen-antibody complexes formed on cancer cells generate cytotoxic immune responses. Cancer cells are abnormal host cells and express large number of tumor antigens. The identification and characterization of tumor antigens gave birth to personalized cancer therapies as it helped define and select appropriate monoclonal antibodies to generate specific and sensitive cytotoxic immune responses. Antibodies are manufactured by drug industry using hybridoma technology to generate highly specific monoclonal antibodies. Macor P. and Tedesco F. in “Complement as an effector system in cancer immunotherapy” in Immunology Letters 111, 2007 6-13 have summarized the importance of host Classical complement system as an effector system in monoclonal antibody therapy of cancers. The examples of such successful cancer therapies are: Hematological malignancies include CD20 and CD22 for B-cell non-Hodgkin's lymphoma; CD33 for acute myeloid leukemia; and CD52 for chronic lymphocytic leukemia.

Solid tumors therapy examples include human epidermal growth factor receptor 2 (HER2, Her-2/neu or c-erbB-2) for breast cancer, epidermal growth factor receptor (EGFR) for colorectal or lung cancer, carcinoembrionic antigen (CEA) for gastrointestinal cancer, epithelial cell adhesion molecules (EpCAM or 17-1A) for colorectal cancer, CA72-4 (TAG-72) for gastrointestinal cancer, high-molecular weight melanoma-associated antigen (HMW-MAA) for malignant melanoma.

Standard treatment options for breast cancer were discussed in the article “Harnessing the Immune System for the Treatment of Breast Cancer” by X. Jiang in the Journal of Zheijang University—Science B (Biomedicine and Biotechnology) 2014 15(1):1-15.

As shown in above review article, the need to improve cytotoxic immune responses is continuous. Several incremental technology advances have been described that improve structural and functional properties of monoclonal antibodies to better engage tumor antigens. The affinity, binding characteristic and cross reactivity features of antigen-antibody complexes can be improvised that can potentially enhance immune targeting capacities of monoclonal antibodies. Genomic advances have led to the new searches for better tumor antigens that can be targeted. Limiting factors for better cancer therapy is expression of Complement regulatory proteins (CRPs) on the surface of numerous cancer cells and cell lines. They control Complement activation acting at different steps of the Complement cascade and restrict assembly of membrane attack complex to induce cytotoxic immune responses.

Another article discusses complement inhibitory proteins that may hamper the clinical efficacy of cancer immunotherapy strategies based on the use of monoclonal antibodies. See “Control of Complement Activation by Cancer Cells and its Implications in Antibody-Mediated Cancer Immunotherapy” by R. Pio, Immunologia, Vol. 25, Num. 3, July-September 2006: 173-187. The article further states that some attempts have been made to modulate antibody-mediated complement activity. In in vitro and in vivo studies, protection by complement regulatory proteins has been overcome by inhibiting their activities or their expression by the target cells.

Therefore, one of the key problems to solve in exploiting complement as an effector system in cancer immunotherapy is to neutralize the inhibitory effect of complement regulatory proteins which are often over expressed on tumor cells. This represents a mechanism of evasion of these cells from complement attack. In one such approach, this situation can be overcome by using neutralizing antibodies to target onto tumor cells together with the specific antibodies directed against tumor specific antigens. This is an area of active investigation and the initial experimental data that start to be available seem to be promising. Monoclonal targeting of the regulatory molecule has been successfully demonstrated as a proof of concept to induce and enhance cytotoxic immune responses. The dominant role of circulating immune regulatory proteins to protect host cells against activated immune system is slowly being recognized.

This aspect is reflected in several publications involving studies in different types of cancers as under.
a. Chronic Lymphocytic Leukemia: S Horl et al studied “Reduction of complement factor H binding to CLL cells improves the induction of rituximab-mediated complement-dependent cytotoxicity” in Leukemia, 2013. 27. 2200-2208.
b. B Cell Lymphoma: Caudwell V. Et al studied “Complement alternative pathway activation and control on membranes of human lymphoid B cell lines” and published their findings in Eur J immunol. 1990 December; 20(12):2643-50.
c. Colon Cancer: Wilczek E et al studied “The possible role of Factor H in colon cancer resistance to complement attack” and published their findings in Int. J. Cancer: 122, 2030-2037 (2008).
d. No-Small Lung Cancer: Ajona D. et al studied “Down-Regulation of Human Complement Factor H Sensitizes Non-Small Cell Lung Cancer Cells to Complement Attack and Reduces in Vivo Tumor Growth” and published their findings in The Journal of Immunology, 2007, 178: 5991-5998.
e. Glioblastoma: Junnkkala S. et al studied “Exceptional resistance of human H2 Glioblastoma cells to complement-mediated killing by expression and utilization of Factor H and Factor H like protein 1” and published their findings in J Immunol. 2000; 164(11):6075-81.
f. Thyroid Cancer: Yamakawa M et al studied “Protection of Thyroid Cancer Cells by Complement-regulatory Factors” and published their findings in Cancer, 1994:73: 2808-17.
g. Ovarian Cancer: Junnikkala S. et al studied “Secretion of soluble complement inhibitors Factor H and Factor H-like protein (FHL-1) by ovarian tumor cells” and published their findings in “British Journal of Cancer (2002) 87, 1119-1127.
h. Cancer Metastasis: Fedarko N. S. et al studied “Factor H Binding to Bone Sialoprotein and Osteopontin Enables Tumor cell Evasion of Complement—Mediated Attack” and published their findings in The Journal of Biological Chemistry. 2000; Vol. 275, No. 22, 16666-16672, 2000.
i. Corey M. J. et al “Mechanistic studies of the Effects of Anti-Factor H Antibodies on complement-mediated lysis” in The Journal of Biological chemistry Vol. 275, No. 17, pp. 12917-12925, 2000.
j. WIPO Publication WO 2011/113641 entitled Complement Factor H for Oxidative Stress Disease Conditions, based on PCT Application PCT/EP2011/051652.

Korbelik M and Cecic I in “Complement Activation Cascade and its regulation: Relevance of Solid tumors to photodynamic therapy” Journal of Photochemistry and Photobiology, 93, 2008, 53-59 detailed a novel approach to target immune regulatory molecules with monoclonal antibodies and photodynamic therapy (PDT). In summary, this work demonstrates that PDT dampens the expression of membrane based Complement Regulatory Proteins (mCRPs) on the surface of treated tumor cells that leaves them more vulnerable to complement attack. Further amplification of this effect by using mCRP-neutralizing antibodies as PDT adjuvant can be exploited for therapeutic gain. Modulating the action of other regulators of complement activity also appears to be a promising approach within this type of combined treatment. From the clinical standpoint, effective PDT and immunotherapy combination modalities offer encouraging prospects, particularly for controlling both local and systemic recurrence of treated cancer.

Not addressed in above studies are the need to control adverse effects of cancer therapies such as host inflammation, cytokine storm and tumor lysis syndrome. The successful targeting of cancer cells can cause life threatening accumulation of divalent ions such as potassium and calcium as well high uric acid and phosphorus. C. Scott et al in “The Tumor Lysis Syndrome” published in N Eng J Med, 2011, 364(19), 1844-1854 details the current clinical management.

Not addressed in above literature is the need to harness the potential power of Alternate complement system to maximize cytotoxic potential for cancer therapy. U.S. Pat. No. 6,805,857 titled “Method of modulating factor D, factor H and CD4 cell immune response with a polystyrene sulfonate, alginate, and saline infusion solution” details a method and formulation to target immune modulating proteins of Alternate complement system. U.S. Pat. No. 5,976,780 titled “Encapsulated cell device” details a method to encapsulate cells using sulfonic polymers in alginate polymer.

Not addressed in above literature is the need to develop appropriate formulation method to selectively target cancer therapy. The need to target cancer cells by both passive methods (Leaky vessels, tumor microenvironment and local application) as well as by active methods (Carbohydrate, receptor and Ab targeted) is highlighted by R. Sinha et al in “Nanotechnology in Cancer Therapeutics: Bioconjugated Nanoparticles for Drug Delivery” published in Mol Cancer Ther 2006 5(8): 1909-17. Innova Bioscience's “Guide to Antibody Labeling and Detection” from July 2010 details generic method for labeling antibody with nanoparticles. It is of interest to note that Voigt J et al in “Differential Uptake of Nanoparticles by Endothelial Cells through Polyelectrolytes with Affinity for Caveolae” published in PNAS, 2014, 111(8), 2942-2947, highlights the Nanoparticles (NPs) can serve as containers for the targeting of therapeutics to tumors. Tumors comprise many cell types including endothelial cells that form the blood vessels. Developing new strategies to target information preferentially to endothelial cells can have major implications in the development of targeted therapeutics. They have discovered that charged polymers containing aromatic sulfonate have pronounced affinity for caveolae, which are highly expressed by endothelial cells. By engineering the surface of lipid NPs to bear sulfonate-containing polymers, lipid NPs that are preferentially taken up by endothelial cells have been demonstrated.

Not addressed in cancer vaccine literature is the need to target immune evasion mechanism of cancer such as factor H. This deficiency is highlighted by Thomas S. N. et al in “Engineering Complement Activation on Polypropylene Sulfide Vaccine Nanoparticles” published in Biomaterials 32 (2011) 2194e2203.

### SUMMARY OF THE INVENTION

It is the object of the invention to harness the power of Alternate complement system to maximize the cytotoxic potential by
a. amplifying classical and lectin based complement system to stabilize C3 Convertase on cancer cell surface, and
b. stimulate membrane attack complexes on cancer cells.

It is another object of the invention to target highly glycosylated Immune regulatory binding site on cancer cells to prevent immune evasion of cancer cells and to stimulate vaccine responses

It is a further object of the invention to enhance safety of cancer therapies by inhibiting complement intermediary proteins such as C3a-C5a that potentially contribute to host inflammation, cytokine storm and tumor lysis syndrome.

Above modes of inventions are best carried out by

a. Nanoengineering polymers to impart functionalized properties to activate complement system. Activation of Alternate complement system is preferred due to the ability of this system to magnify activation of classical and lectin based system.

b. Nanoengineering polymers further to target immune evasion mechanism such as Factor H or glycosylated common denominator of immune regulatory proteins of q32 proteins in chromosome 1.

c. Developing formulation method where drug-Ab conjugate synergistically and actively target cancer antigens and its immune evasion properties.

d. Developing formulation method where the drug-Ab conjugate is facilitated to penetrate microenvironment of cancer cells and its vasculature.

It is additional object of the invention to develop formulation variations where the desired monoclonal antibody is combined with functionalized nanopolymer to facilitate personalized therapeutic targeting of three dimensional interactions of host immune system with monoclonal antibodies in different types of cancers.

In one embodiment the invention relates to a method involves combining existing and evolving cancer therapeutics with functionalized nanopolymers either at manufacturing level or at bedside to maximize cytotoxic potentials.

The method further involves combining functionalized sulfonated polymers with current and evovling cancer vaccines to target immune evasion mechanism of cancer to improve vaccine potentials to maximize cytotoxic potentials.

Another aspect of the invention involves functionalized sulfonated nanopolymers combined with natural polymer such as ultrapurified alginate to form coated particles that can be retained in ex-vivo device so that in one mode it can be used as therapeutic device to inhibit host inflammation, cytokine storm as well as tumor lysis syndrome.

Alternately it can be used as ex-vivo testing device to evaluate adverse effects of new cancer drugs when combined with sulfonated polymers and compare them with device without sulfonated polymer. Such testing require circulating patient's blood sample through device and doing blood test of various inflammatory cytokines and electrolytes.

The concepts and objects described herein are carried out in a first embodiment by a method of providing personilized cancer therapy. The first step involves nanoengineering an inert polymeric compound, for example, styrene, ethenylbenzene, vinyl benzene or phenylethene. The nanoengineered inert polymeric compound is sulfonated to provide a functionalized sulfonated nanopolymer to harness the power of Alternate complement to stimulate and amplify classical and lectin based system to generate cytotoxic immune responses. The functionalized sulfonated nanopolymers then selectively target one or more of:

(i) immune evasion mechanism such as Factor H;

(ii) a glycosylated surface of cancer cells having immune regulatory receptors of chromosome 1 at Q32 position; or

(iii) cancer antigens and penetration of tumor microenvironment by combining the functionalized sulfonated nanopolymers with a monoclonal Ab and a cancer drug to form a drug-Ab conjugate to provide personalized cancer therapy.

The nanoengineering step further includes delivering the inert polymeric compound as beads; and fractionating the beads to form particles of less than 100 nanometers in diameter. Following the sulfonating step, the method further includes purifying the functionalized sulfonated nanopolymer by dialysis. Also following the sulfonating step, the method further includes reformulating the nanoformulated functionalized sulfonated nanopolymer to selectively target cancer cells to maximize its cytotoxic potential in the blood and at tissue levels.

The selectively targeting step (ii) further includes selectively targeting a glycosylated surface of cancer cells for inhibiting inflammatory cytokines liberated due to C3a-C5a complement breakdown products for enhancing safety of cancer therapy. The steo of enhancing safety additionally includes enhancing safety of cytotoxic cancer therapy by reducing the amount of functionalized sulfonated nanopolymer and gelling and localizing the functionalized sulfonated nanopolymer at cancer tissues. The enhancing safety step additionally includes enhancing safety of cytotoxic cancer therapy by reducing the amount of functionalized sulfonated nanopolymer and gelling the functionalized sulfonated nanopolymer in blood by an ex-vivo device for inhibiting inflammatory cytokines and removing divalent toxins generated due to tumolysis syndrome.

After the sulfonating step, the method further includes retaining the functionalized sulfonated nanopolymer in an ex-vivo device for inhibiting host inflammation due to cytokine storm and removing divalent toxins as in tumor lysis syndrome. Following the retaining step, the method further includes circulating monoclonal antibodies against cancer cells through the ex-vivo device for contacting the functionalized sulfonated nanopolymer for evaluating adverse effects of new cancer drugs. After the retaining step, the method includes circulating a patient's blood through the ex-vivo device; testing the circulated blood for inflammatory cytokines and electrolytes; and evaluating toxic potentials of cancer monoclonal antibodies.

Following said sulfonating step, the method further includes combining cancer vaccines with functionalized sulfonated nanopolymers for targeting immune evasion mechanism of cancer for improving vaccine potentials for maximizing cytotoxic vaccine potentials. The nanoengineering step further includes combining the particles with one of natural polymers and ultrapurified alginate to form coated particles

### BRIEF DESCRIPTION OF THE DRAWINGS

The advantages, nature, and various additional features of the invention will appear more fully upon consideration of the illustrative embodiments now to be described in detail in connection with accompanying drawings. In the drawings wherein like reference numerals denote similar components throughout the views:

FIG. 1 is a diagram showing various relationships to immune regulatory proteins.

FIG. 2 is a diagram illustrating the classical pathway which is used for current monoclonal antibody therapy.

FIG. 3 is a diagram illustrating the classical pathway and the alternate pathway.

FIG. 4 is a diagram illustrating an approach according the invention involving classical, lectin and alternate pathways.

FIG. 5 is a further diagram showing classical, lectin and alternate pathways.

FIG. 6 is a graph plotting residual Factor D & Factor H versus time.

FIG. 7 is a diagram showing the relationship of Factor D & Factor H with the 1q32 protein.

FIG. 8 is a molecular diagram of purified alginate.

FIG. 9 is a schematic diagram of a micro-encapsulator device.

FIG. 10 is an illustration of droplet production.

FIG. 11 is a molecular diagram of the final structure of the coated nanopolymer.

FIG. 12 is an illustration of an ex vivo device.

### DETAILED DESCRIPTION OF THE PREFERRED EMBODIMENTS

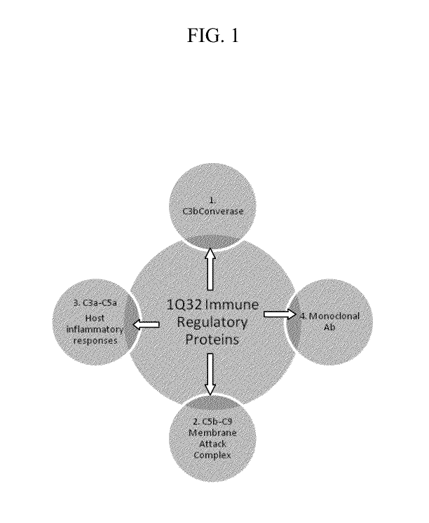

These and other aspects of the invention will be obvious to the skilled physician or oncologists involved in the art of caring for cancer patients by careful perusal of FIG. 1. More particularly, the central globe in the diagram FIG. 1 refers to the 1Q32 Immune Regulatory Proteins. At the 12:00 position is the first, 1. C3b Converase. In the 6:00 position is the second, 2. C5b-C9 Membrane Attack Complex. In the 9:00 position is the third, 3. C3a-C5a Host Inflammatory Responses. In the 3:00 position is the fourth, 4. Monoclonal Ab.

The presence of immune regulatory molecules on cancer cell surface interferes with cytotoxic potential of monoclonal antibodies, as they contribute to the following:

a. Destabilization of C3b Convertase;

b. Suboptimal formation of C5b-C9 membrane attack complex;

c. Stimulation of host inflammation due to formation of C3a-C5a and its downstream inflammatory cytokines; and

d. Inefficient targeting with Monoclonal antibody in cancer therapy increase cost, adverse effects and contribute to increased morbidity and mortality due to cancer resistance and its spread.

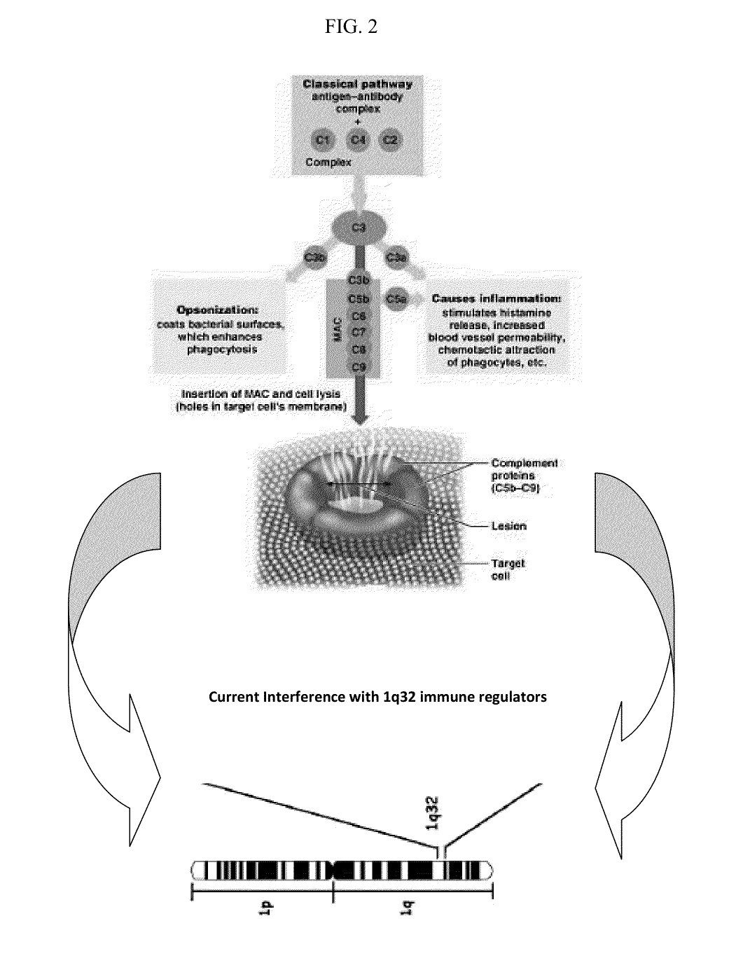

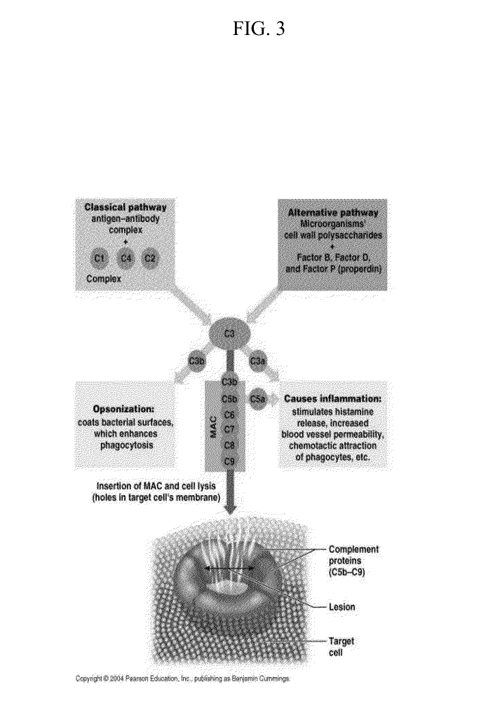

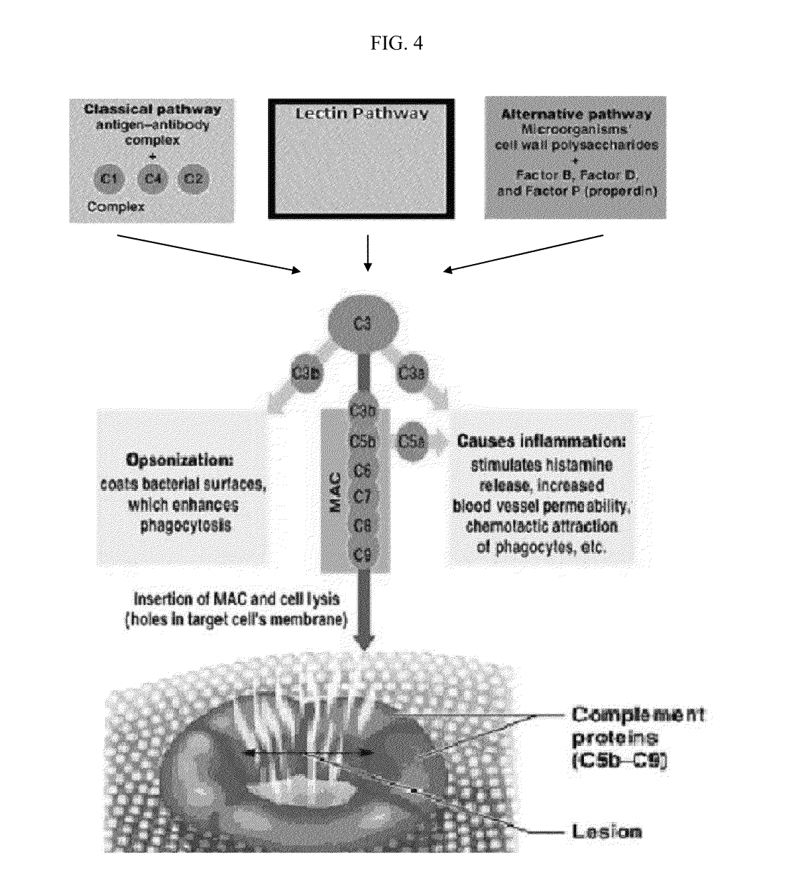

There are three players in complement based effector immune responses: Classical, Lectin and Alternate. See FIGS. 2, 3 and 4. They are primed proximally to generate C3B Convertase, flagging molecules on foreign cancer cells. Monoclonal antibodies that form tumor antigen-Antibody complexes on cancer cell surface engages and amplifies mainly classical complement system to induce cytotoxic immune responses. The cytotoxic power of host lectin and alternate pathway system is not efficiently utilized to complement cytotoxic potential of classical system. The presence of immune regulatory molecules on cancer cell surface and failure to use Lectin and Alternate complement system efficiently leads to suboptimal responses. This relates to the fact that cancer flagging molecule such as C3 Convertase is not stabilized on cancer cells due to the neutralizing effect of immune regulatory molecules on C3 Convertase. Stability of C3 Convertase is critical step to generate C5b-C9 immune responses. Failure to stabilize C3 Convertase on cancer cell surface leads to poor effector cytotoxic immune responses. The Suboptimal responses are complicated by increased host inflammation. Zhang X et al in “Regulation of Toll-like receptor-mediated inflammatory response by complement in vivo” Blood, 2007, 110(1), 228-236, detail the underlying mechanism. When “Serine Protease” of complement system is activated it leads a series of downstream chain reactions. For example, there is constant generation of C3a and C5a fragments. These are chemotactic and anaphylactic fragments. They stimulate Toll Receptors functions of TLR4, TLR2, TLR 6 and TLR 9 in cells. This liberates inflammatory cytokines such as IL-1, IL-6 and TNF alfa. The current therapy of “Cytokine storm” requires hospitalization, intensive medical care of patient and measures are largely supportive. They are directed to maintain fluid and electrolyte balance, monitoring of vital signs, use of respirator and steroids. This is associated with high mortality. The successful targeting of cancer cells can cause life threatening accumulation of divalent ions such as potassium and calcium as well high uric acid and phosphorus. Rampelli E. et al in “The management of Tumor-Lysis” syndrome” published in Nat. Clin. Pract. Oncol. 2006, 3(8), 438-447, details the current clinical management.

The classical complement system when activated, illustrated in FIG. 2, forms C3 and then C3b Convertase. C3b Convertase is a flagging molecule that is stabilized on top of cancer cell surface. This stability is essential to initiate cytotoxic immune responses by generating membrane attack complex. Properdin is a positive immune regulator to this effect. (Kouser L E al in “Properdin and Factor H: Opposing Players on the alternative complement pathway “See-Saw” Review Article in FIMMU, 2013:Vol 4, 1-10). This initiates formation of Membrane attack complex through assembly of C5b-C9 Complex. As shown in the above article, Factor H and its variants is a negative immune regulator.

The human genes encoding the regulatory complement components C4-binding protein (C4BP), the Cab/C4b receptor (CR1), the decay-accelerating factor (DAF), and factor H (H) are linked and define the regulator of complement activation (RCA) gene cluster, which maps to band q32 of chromosome 1. The same chromosomal location has been reported for the human gene encoding the C3dg receptor (CR2), suggesting that CR2 also belongs to this linkage group. Since the RCA gene cluster encodes the proteins involved in the control of the C3-convertases, it represents the regulatory counterpart of the class III gene cluster of the MHC that encodes the structural components of the C3-convertases C2, B, and C4. (Campos J R, Rubinstein P and Cordoba S R in A Brief Definitive Report: ‘A Physical Map of the Human Regulator of Complement activation gene cluster linking the complement genes CR1, CR2. DAF and C4BP in J. Exp. Med., 1988, Volume 167, 664-669 and also, Gordillo J. E. et al “Predisposition to atypical hemolytic uremic syndrome involves the concurrence of different susceptibility alleles in the regulators of complement activation gene cluster in 1q32 in Human Molecular Genetics, 2005, Vol 14:5, 703-712).

In summary, the data from experimental models of cancer and clinical studies suggest that modulating Complement regulatory proteins on cell surface of tumor cell has the potential to increase therapeutic efficacy of a mAb by triggering Complement dependent effector mechanisms.

The recent advances in genomics and fundamentals of immunology have recognized dominant role of Factor H as an immune regulatory molecule. Factor H is a known immune regulatory molecule of Alternate complement system. It is also the immune regulatory molecule on host cell surface that regulates classical, lectin and alternate pathway system to prevent immune activation on host cell surface. When, Factor H is over secreted or expressed on cancer cells, it is a negative regulator of C5b-C9 complex and proximally destabilizes the formation of C3b Convertase. This will protect cancer cells like normal host cells and cause inflammatory host responses by C3b Convertase inactivation that forms C3a and C5a inflammatory molecules.

Several papers are published in recent years highlighting the dominant role of Factor H expression in tumor cells and ability of Factor H antibodies to induce cytotoxic immune responses. The dominant role of circulating immune regulatory proteins to protect host cells against activated immune system is slowly being recognized. In evaluating the structure-function relationship of circulating immune regulatory proteins with membrane bound proteins, a study of genes involved in the regulation of complement activation (RCA) is particularly instructive. Both membranes bound and circulatory immune regulatory proteins are structurally related. The common denominator is the high level of glycosylation.

Functionally, a careful perusal of circulating and membrane immune regulators show that Factor H is a key immune regulator, Factor I is a cofactor and the later is intimately involved in the functional regulation of membrane immune regulators. Membrane immune regulatory factors engage Factor I for subsequent complement product inactivation. In keeping with this recent advances in immunology, are numerous reports showing expression of Factor H and its related molecules on cancer cell surface. The proof of concept data is generated by targeting immune regulatory molecule with Factor H antibody to stimulate cytotoxic immune responses. However, better response is expected by targeting the immune regulatory molecules that have common properties of being highly glycosylated.

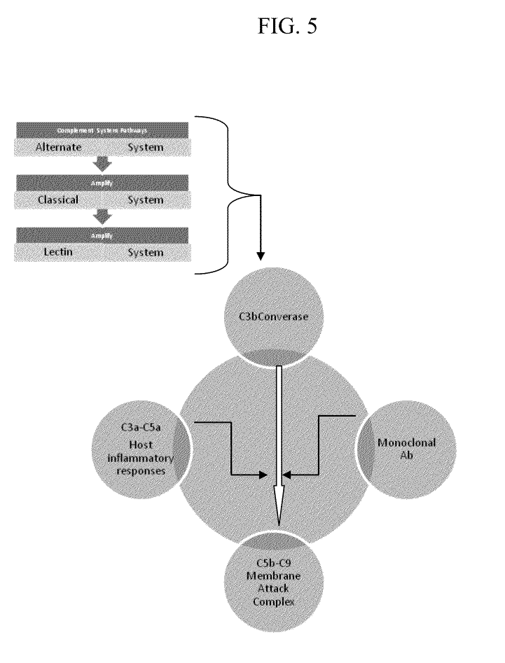

Details of how the therapy according to the invention is carried out will be obvious from FIG. 5 which diagrams the impact of targeting immune regulatory molecules of 1Q32. This also illustrates inhibiting of negative immune regulators of Complement System (1Q32) proteins which allows Properdin to positively regulate the assembly of C3B Converase and stabilize it on the cancer cell surface. This also activates Alternate Complement System which is an amplifier of both lectin and Classical Complement System. The specificity and sensitivity of monoclonal antibodies will improve and will lead to reduce nonspecific inflammatory responses.

Below is evidence that functionalized sulfonated polymers target

a. Immune regulatory molecule to stimulate cytotoxic immune responses against cancer cells. A representative molecule for targeting selected is Factor H which is highly glycoslylated.

b. Inhibiting adverse effects of cytokine storm and host inflammatory responses in the host. Here the representative molecule for targeting selected is C1q of classical complement system and Factor D of alternate complement system.

### In-Vitro Data:

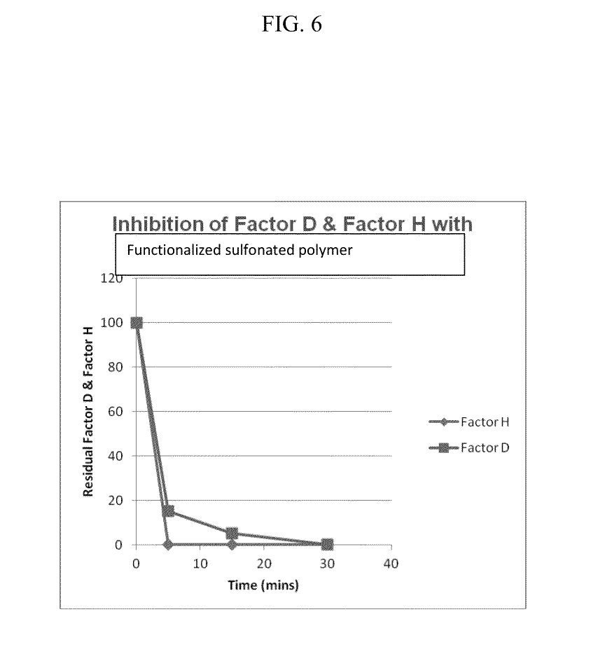

When in in-vitro experiments fresh Normal Human Serum in 1/10 dilution is contacted with functionalized polymer and incubated for 30 minutes at 37*C, the drug inhibit Factor H and Factor D. The Factor H inhibition is twice stronger (30 mg dose) than Factor D (60 mg dose). Thus 100% factor H inhibition in-vitro occurs at 30 mg dose. While 100% factor D inhibition as well as Factor H occurs at 60 mg. FIG. 6 is a graph showing Inhibition of Factor D & Factor H with a Functionalized Sulfonated Polymer. The graph plots Residual Factor D & Factor H levels with respect to time. The top line represents Factor D and the bottom line represents Factor H. Similarly, at higher dose, C1q is inhibited to prevent activation of classical complement system.

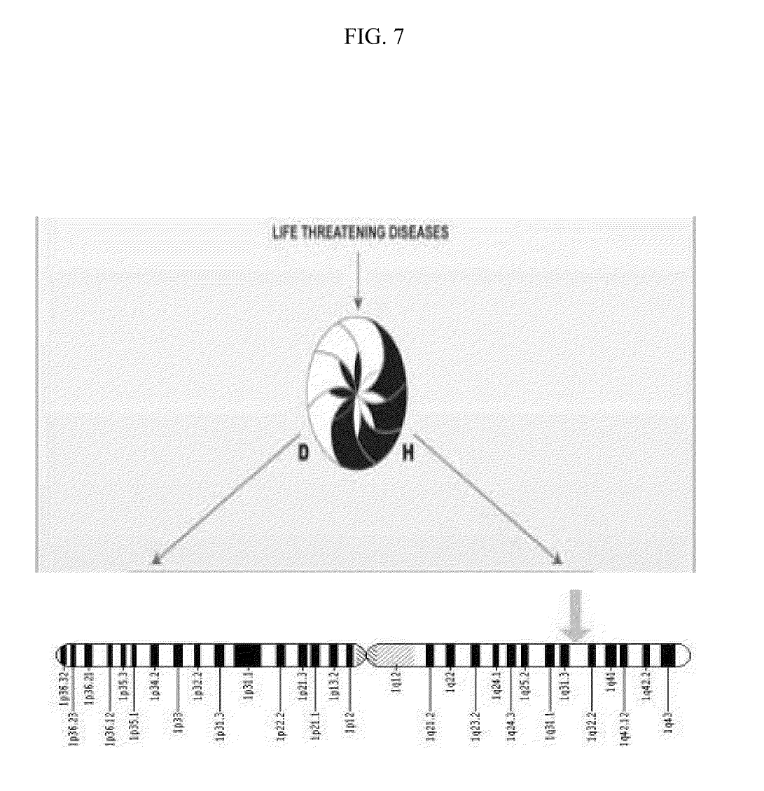

As shown in the FIG. 7, the drug targets 1q32 protein that are highly glycosylated, in particular, Factor H to stimulate cytotoxic responses. The diagram illustrates the following:

a. Factor H in Cancer over secreted,

b. Cancer Cells are immune protected

c. Cancer resistance to therapeutics.

These effects can be overcome by efficient targeting of highly glycosylated complement regulatory proteins where Factor H is a dominant player.

### Manufacturing Procedure of Purified, Ultra Small Sodium Polystyrene Sulfonate

The manufacturing process uses a sodium polystyrene sulphonate resin. One commercially available product is Purolite, an ion exchange resin, designated as sodium polystyrene sulphonate resin and sold under the tradename Purolite C-100 MR.

The product description indicates that Purolite C-100 MR is a strong acid cation exchange resin powder. The pharmaceutical grade product is used for the treatment of specific medical conditions. In certain cases there can be a buildup of potassium in the bloodstream which is not removed by the kidneys. The function of the resin is to reduce and control, where necessary, the potassium levels within the body. Typical physical and chemical characteristics include:

| | |
|---|---|
| Polymer Matrix Structure | Cross-linked gel polystyrene |
| Smell and Appearance | Odorless buff powder |
| Functional Groups | R—SO₃ |
| Ionic form - as shipped | Sodium - Na |
| Potassium Exchange Capacity | 110-135 mg/dry g |
| Moisture Uptake (Chemical) | 46-54% |
| Moisture Content - as shipped | 10% max |
| (loss on drying) | |
| Particle size range (microns) | <150 |
| Sodium Content (dry resin basis) | 9.4-11.0& |
| Residual Potassium (max) | 0.1% |
| Residual Calcium (max) | 0.1% |
| Iron Content as ppm by Weight (max) | 0.1% |
| Lead Content as ppm by Weight (max) | 0.1% |
| Total Aerobes | <100 |
| Total Yeast | <10 |
| Total Moulds | <10 |
| Others | Not Detectable |

Step 1: obtain sodium polystyrene which is an existing therapeutic drug approved by FDA.

Step 2: The commercial available drug has many impurities. It is subjected to purification by dialysis utilizing the modification of procedure detailed by Sen A. K. et al detailed in “On the importance of purification of Sodium Polystyrene Sulfonate” published in Analytical Chemistry, 2012, 514509, Pages: 1-5.

Step 3: Nanopolymers are formed by using a modification of procedure detailed by Brijmohan S. B. et al in “Synthesis and characterization of Cross-linked Sulfonated Polystyrene nanoparticles” published in Ind. Eng. Chem. Rese. 2005; 44, 8039-8045

An example of functionalized nanopolymer and its characterization is as under.

Sodium Polystyrene Sulfonate Nanopowder

Purity: 99.9%, APS: <100NM, Powder Stock No. NS6130-1 1-000044.

Sodium polystyrene sulfonate nanopowder is a strong acid cation exchange resin powder. This is a pharmaceutical grade product which is used for the treatment of specific medical condition.

Technical Specifications

| Total Capacity (Minimal) | 110-135 mg/dry gram, potassium form | |
|---|---|---|
| Moisture uptake Chemical) | 48-56% | |
| Moisture content | 10% | |
| Average Particle Size | <100 | nm |
| Sodium Content | 9.4-10.5% | |
| Residual Calcium | 0.1% | |
| Iron Content (Maximum) | 90 | ppm |
| Lead (Maximum) | 8 | ppm |

| | |
|---|---|
| Total Aerobes | <110 |
| Total Yeast | <9 |
| Total Moulds | <8 |
| Residual potassium (Maximum) | <0.2% |
| Temp Limit (Stability) | 212 Degree C. |

Basic Features:
Application: Sodium Polystyrene Sulfonate
Polymer Structure: Cross Linked Gel Polymer
Appearance: Odorless buff Powder
Functional Group: Sulfonic Group
Ionic form as shipped: Sodium

Below we detail its formulation variation that can be most effectively used for targeted cancer therapy in conjunction with available and evolving cancer therapies.

Nanoparticles by reducing the particle size will provide increase surface area, are easier to be absorbed from mucus surface permitting oral or nasal formulations for delivery in conjuction with parenteral monoclonal antibodies. The later may be given independent of combing them with formulation methods. In another mode of formulation, Nanoparticles are conjugated with patient's Red Blood Cells or donor matched Red Blood Cells using a modification of procedure detailed by Hu C M J et al in “Erythrocyte membrane-camouflaged polymeric nanoparticles as a biomimetic delivery platform” published in PNAS, 2011: 108, No. 2, 0980-0985. Such nanoparticles conjugated with RBC that was described in above articles essentially are inert and acted as carrier molecule to attach monoclonal antibodies. However, the use of sulfonated, functionalized nanopolymers are active therapeutic compounds that can be additionally linked with monoclonal antibodies to synergize cytotoxic immune responses. Nanopolymers by improving surface areas can reduce the quantity of therapeutic dose and will provide better tumor penetration to improve cytotoxic potential of monoclonal antibodies.

The step of nanoengineering an inert polymeric compound involves providing a styrene monomer and polymerizing to form a nanopolymer, i.e. a polystryrene in nanoparticlulate form. Alternatively, an ethenylbenzene, vinyl benzene or phenylethene may be provided as the starting material and polymerized to form a polymer in nanoparticulate form. These polymers in nanoparticulate form are collectively referred to as nanoengineered polymeric compounds.

The sulfonating step involves introducing a sulfonic group (SO₃H) into the nanoengineered polymeric compounds to provide a functionalized sulfonated nanopolymer. The functionalized sulfonated nanopolymer harnesses the power of Alternate complement to stimulate and amplify classical and lectin based system to generate cytotoxic immune response.

The functionalized sulfonated nanopolymer is then used to selectively target immune evasion mechanism such as Factor H. Alternatively, the functionalized sulfonated nanopolymer is then used to selectively target a glycosylated surface of cancer cells having immune regulatory receptors of chromosome 1 at the Q32 position.

A variation of above formulation method may involve attaching carboxylated functional group and then attach desired monoclonal antibody. More particularly, the functionalized sulfonated nanopolymer is combined with a monoclonal Ab and a cancer drug to form a drug-Ab conjugate to selectively target cancer antigens and penetration of tumor microenvironments to provide personalized cancer therapy. Alternatively, the functionalized sulfonated nanopolymer is combined with cancer vaccines for targeting immune evasion mechanism of cancer for improving vaccine potentials for maximizing cytotoxic vaccine potentials.

Below nanopolymer is coated with ultra purified natural polymer to develop ex-vivo or in-vitro cartridge.

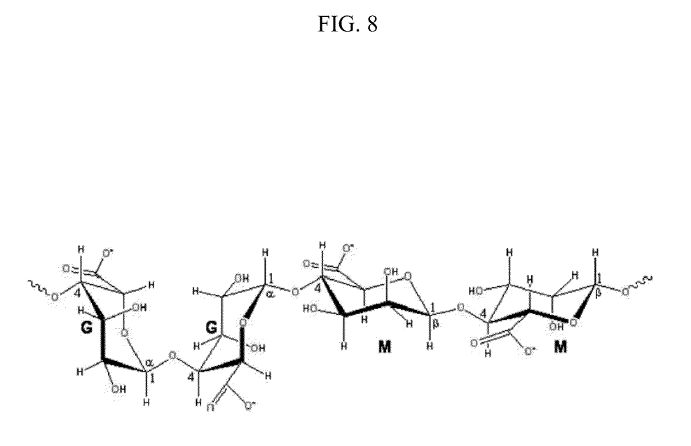

Step 1: The nanopolymer particles are combined with natural polymers and ultrapurified alginate to form coated particles. More specifically, the particles are modified by coating its surface with ultra purified alginate using 0.1% solution. The resulting structure of alginate material is illustrated in FIG. 8, where G is a Guluronic acid group, and M is a Manuronic acid group. The preferred structure is ultra-purified alginate rich in G polymer.

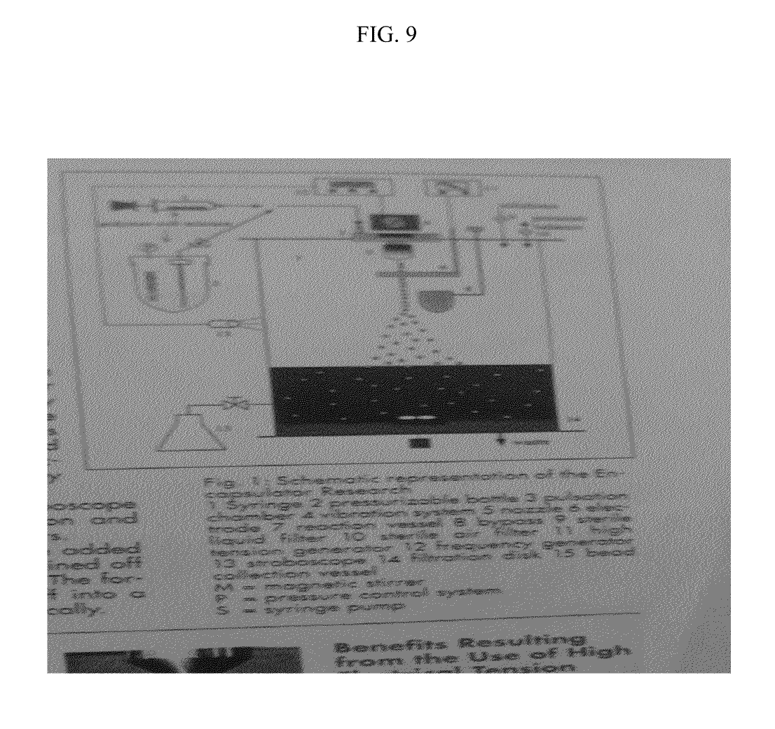

Step 2: The mixture is passed through a micro-encapsulator device, a schematic diagram of which is shown in FIG. 9. A syringe 1 and pressurizable bottle 2 deliver two different materials to pulsation chamber 3. A vibration system 4 causes the combined materials to exit via nozzle 5 as the material is attracted to electrode 6. The material is delivered to the reaction vessel 7 equipped with a bypass 8. The vessel also includes a sterile liquid filter 9 and a sterile air filter 10. A high tension generator 11 is coupled to the electrode 6. A frequency generator 12 is coupled to the vibration system 4 and a stroboscope 13. A filtration disk 14 is provided at the bottom of vessel 7. A bead collection vessel 15 is connected to vessel 7 to capture beads that have been removed from the vessel. M refers to a magnetic stirrer. P refers to a pressure control system. S refers to a syringe pump.

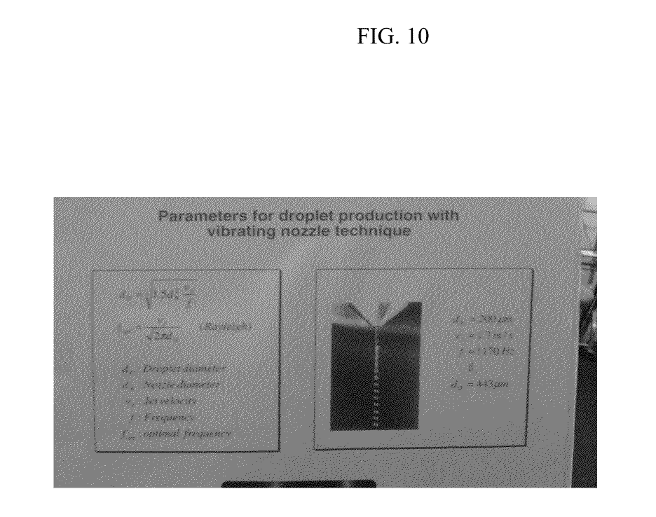

Step 3: Using a vibrating nozzle, the drops are accumulated on top of calcium chloride solution (0.1%) in 0.9% sodium Chloride using Raleigh's formula, as shown in FIG. 10.

In Controlling the variable of droplet production, the main Formula 1 is:

$${d}_{D} = \sqrt[3]{1.5 {d}_{N}^{2} \frac{{v}_{j}}{f}} \qquad Formula 1$$

We know from Rayleigh Formula 2, that the optimal frequency is calculated by:

$${f}_{opt} = \frac{{v}_{j}}{\sqrt{2} π {d}_{N}} \qquad Formula 2$$

Where the variables are defined as follows
d_{D}: Droplet diameter
d_{N}: Nozzle diameter
v_{J}: Jet velocity
f: Frequency
fₒₚₜ: optimal frequency

In a practical example, the nozzle diameter, jet velocity and frequency were set as follows:

d_{N}=200 μm

v_{J}=1.7 m/s

f=1170 Hz

The resulting droplet diameter was as follows:

d_{D}=443 μm

In other words, nanoengineering further includes delivering inert polymeric compound beads and fractionating the beads to form particles of less than 100 nanometers in diameter. The functionalized sulfonated nanopolymers may be purified by dialysis. The functionalized sulfonated nanopolymers may be reformulated to selectively target cancer cells to maximize its cytotoxic potential in the blood and at tissue levels. Alternatively, the selective targeting may include selectively targeting a glycosylated surface of cancer cells for inhibiting inflammatory cytokines liberated due to C3a-C5a complement breakdown products for enhancing safety of cancer therapy.

Above leads to the formation of swellable synthetic cell membrane that retains sodium in the beads and have capacity to exchange potassium 2.9+/−2 mEq per gram of drug. The artificial cell membrane formed is of diameter from 25 micron to 150 micron. The bead size could be reduced to less than 1 micron size by mixing the content with nanopolymers of sodium polystyrene sulfonate. The newly formulated drug is freeze dried and stored at room temperature until use. The safety of cytotoxic cancer therapy may be enhanced by reducing the amount of functionalized sulfonated nanoploymer and gelling and localizing the functionalized sulfonated nanopolymer at cancer tissues. Alternatively, the safety of cytotoxic cancer therapy may be enhanced by reducing the amount of functionalized sulfonated nanoploymer and gelling the functionalized sulfonated nanopolymer in blood by an ex vivo device for inhibiting inflammatory cytokines and removing divalent toxins generated due to tumolysis syndrome.

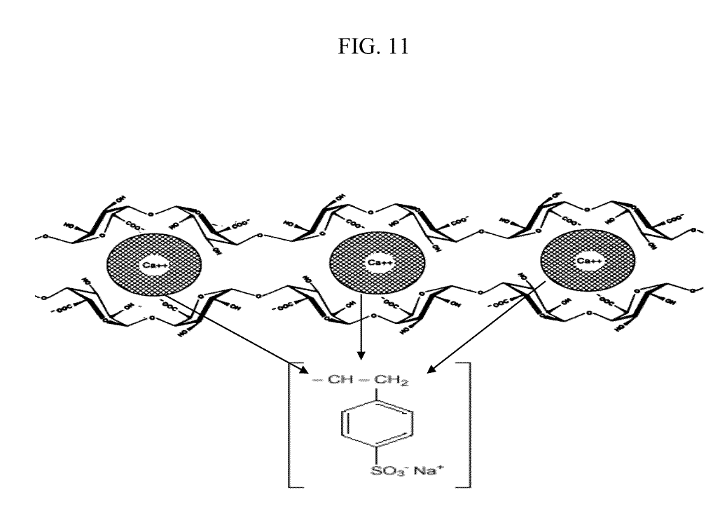

The final structure of coated nanopolymer is illustrated in FIG. 11. The coated nanopolymer can be retained in ex-vivo cartridge.

### Manufacturing and Formulation Variations: Development of Ex-Vivo Device

This object is to reduce adverse effects of chemotherapy and monoclonal antibodies such as “Tumor-lysis syndrome” and “Cytokine Storm.”

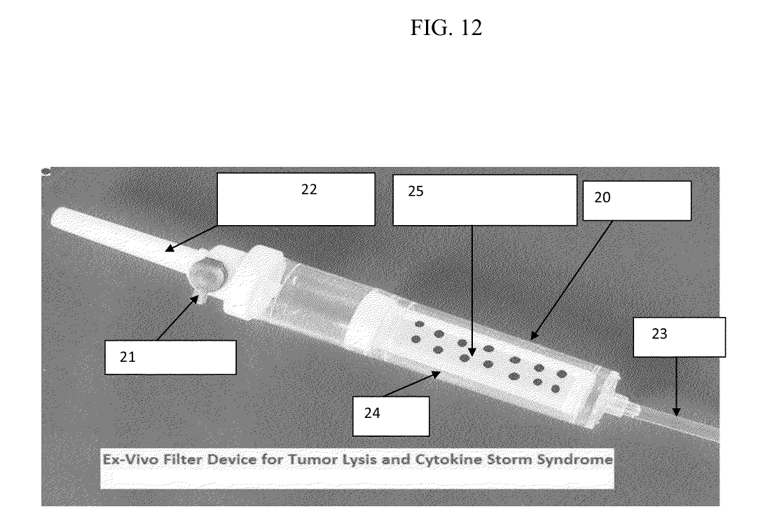

FIG. 12 is an ex-vivo device that can be used to provide minute to minute control of adverse effects and can be tailored to therapeutic needs readily. The device includes a reservoir 20 connected via a port control valve 21 to a connector and filler device 22. The other side of reservoir 20 is coupled to tubing 23. Within reservoir 20 is a filter 24 containing the encapsulated polymer 25.

The manufacturing and formulation protocol is modified from Rousseau I. et al “Entrapment and release of sodium polystyrene sulfonate (SPS) from calcium alginate gel beads” in European Polymer Journal, 2004: 40; pages 2709-2715. In other words, the functionalized sulfonated nanopolymer is retained in an ex vivo device for inhibiting host inflammation due to cytokine storm and removing divalent toxins as in tumor lysis syndrome. Alternatively monoclonal antibodies against cancer cells are circulated throughout the ex vivo device for contacting the functionalized sulfonated nanopolymer for evaluating adverse effects of new cancer drugs.

Above device is connected to blood both proximally and distally. It can be used as in-vitro testing cartridge where A [should this be “a” or “Ab” ] test dose of monoclonal antibody and Immune regulatory sulfonic polymer are added and its adverse effects are monitored by blood drawn from other arm of the device to test for various cytokines and cytotoxic responses generated. Once ascertained that medicine is reasonably tolerated and appropriate for the patient's need, the concentration of drug increased to desirable level. This will provide preventive, safe therapeutic approach. The steps can be itemized as circulating a patient's blood through the ex vivo device, testing circulated blood for inflammatory cytokines and electrolytes, and evaluating toxic potentials of cancer monoclonal antibodies.

During therapy, large cytotoxic effect may precipitate either tumor lysis syndrome or cytokine syndrome. Routine blood chemistry will be instructive to the occurrence of high potassium, Calcium, phosphorus or uric acid along with renal functions. Therapeutic method involve stopping the cytotoxic immune therapy and delivering desired dose of sulfonic polymer to inhibit complement system and remove high potassium and calcium.

Having described preferred embodiments for (which are intended to be illustrative and not limiting), it is noted that modifications and variations can be made by persons skilled in the art in light of the above teachings. In practicing the formulation methods, alternate or additional steps may be included that do not alter the purpose of the invention. The use of equivalent materials other than those specified are intended to be included within the scope of the invention. It is therefore to be understood that changes may be made in the particular embodiments of the invention disclosed which are within the scope and spirit of the invention as outlined by the appended claims. Having thus described the invention with the details and particularity required by the patent laws, what is claimed and desired protected by Letters Patent is set forth in the appended claims.

What is claimed is:

1. A method of providing personilized cancer therapy comprising:
nanoengineering an inert polymeric compound selected from the group consisting of styrene, ethenylbenzene, vinyl benzene and phenylethene;
sulfonating the nanoengineered inert polymeric compound to provide a funtionalized sulfonated nanopolymer to harness the power of Alternate complement to stimulate and amplify classical and lectin based system to generate cytotoxic immune responses; and
selectively targeting with the functionalized sulfonated nanopolymers one of:
(i) immune evasion mechanism such as Factor H;
(ii) a glycosylated surface of cancer cells having immune regulatory receptors of chromosome 1 at Q32 position; or
(iii) cancer antigens and penetration of tumor microenvironment by combining the functionalized sulfonated nanopolymers with a monoclonal Ab and a cancer drug to form a drug-Ab conjugate to provide personalized cancer therapy.

2. The method of claim 1, wherein said nanoengineering step further includes:
delivering the inert polymeric compound as beads; and
fractionating the beads to form particles of less than 100 nanometers in diameter.

3. The method of claim 2, wherein following said sulfonating step, the method further includes:
purifying the functionalized sulfonated nanopolymer by dialysis.

4. The method of claim 3, wherein following said sulfonating step, the method further includes:
reformulating the nanoformulated functionalized sulfonated nanopolymer to selectively target cancer cells to maximize its cytotoxic potential in the blood and at tissue levels.

5. The method of claim 4, wherein said selectively targeting step (ii) further includes:
selectively targeting a glycosylated surface of cancer cells for inhibiting inflammatory cytokines liberated due to C3a-C5a complement breakdown products for enhancing safety of cancer therapy.

6. The method of claim 5, wherein enhancing safety additionally includes:
enhancing safety of cytotoxic cancer therapy by reducing the amount of functionalized sulfonated nanopolymer and gelling and localizing the functionalized sulfonated nanopolymer at cancer tissues.

7. The method of claim 5, wherein enhancing safety additionally includes:
enhancing safety of cytotoxic cancer therapy by reducing the amount of functionalized sulfonated nanopolymer and gelling the functionalized sulfonated nanopolymer in blood by an ex-vivo device for inhibiting inflammatory cytokines and removing divalent toxins generated due to tumolysis syndrome.

8. The method of claim 1, further including after the sulfonating step:
retaining the functionalized sulfonated nanopolymer in an ex-vivo device for inhibiting host inflammation due to cytokine storm and removing divalent toxins as in tumor lysis syndrome.

9. The method of claim 8, further including after the retaining step:
circulating monoclonal antibodies against cancer cells through the ex-vivo device for contacting the functionalized sulfonated nanopolymer for evaluating adverse effects of new cancer drugs.

10. The method of claim 9, further including after the retaining step:
circulating a patient's blood through the ex-vivo device;
testing the circulated blood for inflammatory cytokines and electrolytes; and
evaluating toxic potentials of cancer monoclonal antibodies.

11. The method of claim 1, wherein following said sulfonating step, the method further includes
combining cancer vaccines with functionalized sulfonated nanopolymers for targeting immune evasion mechanism of cancer for improving vaccine potentials for maximizing cytotoxic vaccine potentials.

12. The method of claim 2, wherein said nanoengineering step further includes
combining the particles with one of natural polymers and ultrapurified alginate to form coated particles.
